(19) United States
(12) Patent Application Publication
(10) Pub. No.: US 2017/0020867 A1
(43) Pub. Date: Jan. 26, 2017

(54) USE OF MASITINIB FOR THE TREATMENT OF CROHN'S DISEASE

(71) Applicant: AB SCIENCE, Paris (FR)

(72) Inventors: Alain MOUSSY, Paris (FR); Jean-Pierre KINET, Lexington, MA (US); Colin MANSFIELD, Ecully (FR)

(21) Appl. No.: 15/215,777

(22) Filed: Jul. 21, 2016

(30) Foreign Application Priority Data
Jul. 21, 2015 (EP) 15 306 185.8

(51) Int. Cl. A61K 31/496; A61K 45/06; A61K 31/56

(52) U.S. Cl. CPC A61K 31/496; A61K 31/56; A61K 45/06

(57) ABSTRACT

A method for treating patients afflicted with Crohn's disease, wherein the patients are treated with a tyrosine kinase inhibitor, mast cell inhibitor or c-Kit inhibitor, in particular masitinib, optionally in combination with at least one pharmaceutically active ingredient for treatment of Crohn's disease.

### FIELD OF THE INVENTION

The present invention relates to a method for treating patients afflicted with Crohn's disease, wherein said patients are treated with a tyrosine kinase inhibitor, mast cell inhibitor or c-Kit inhibitor, in particular masitinib, optionally in combination with at least one pharmaceutically active ingredient.

### BACKGROUND OF THE INVENTION

Crohn's disease is a chronic, idiopathic inflammatory bowel disease (IBD), which mechanism involves T lymphocyte and inflammatory cytokine production.

Its incidence seems more important in the United-States and the north of Europe and approximately ranges from 1 to 6 per 100,000 persons. Its prevalence is estimated to be 90 per 100,000 persons. Recent epidemiological studies have found increased mortality risk in patients with Crohn's disease and most individuals experience an impact of the disease on their daily life.

Crohn's disease is characterized by recurring episodes of inflammation of any part of the bowel, from the mouth to the anus. Said inflammation is noncontiguous and thus can produce skip lesions throughout the bowel.

It typically manifests in the gastrointestinal tract and can be categorized by the specific tract region affected. A disease of both the ileum and the large intestine, ileocolic Crohn's, accounts for 50% of cases. Crohn's ileitis, manifest in the ileum only, accounts for 30% of cases, while Crohn's colitis, of the large intestine, accounts for the remaining 20% of cases. Gastroduodenal Crohn's disease causes inflammation in the stomach and first part of duodenum. Jejunoileitis causes spotty patches of inflammation in the jejunum.

Crohn's disease may also be categorized by the behavior of disease as it progresses. These categorizations are formalized in the Vienna classification of the disease. There are three categories of disease presentation in Crohn's disease: stricturing, penetrating, and inflammatory. Stricturing disease causes narrowing of the bowel that may lead to bowel obstruction or changes in the caliber of the feces. Penetrating disease creates abnormal passageways (fistulae) between the bowel and other structures, such as the skin. Inflammatory disease (or nonstricturing, nonpenetrating disease) causes inflammation without causing strictures or fistulae.

Drug treatment has remained the mainstay of treatment for patients with Crohn's disease but surgery is still required in approximately three quarters of patients with Crohn's disease.

The goals of drug treatment for Crohn's disease include induction of remission in patients with active disease and maintenance of remission in those with quiescent disease that is either medically or surgically induced, while improving the patient's quality of life.

Therapies have focused on attenuation of the enteric immune response and inflammatory process. Conventional therapies, such as aminosalicylates, including balsalazide, olsalazine, mesalamin, and sulfasalazine, and corticosteroids, including budesonide, prednisolone, and dexamethasone, continue to be widely used.

These can be described as controller medications, taken to keep the symptoms of Crohn's disease under clinical control, chiefly through their anti-inflammatory effects. Corticosteroid pills or intravenous treatments are used to stop symptoms of moderate to severe Crohn's disease when aminosalicylates have not worked to stop a flare-up of Crohn's disease. However, the systemic side effects of long-term corticosteroid exposure include osteoporosis, arterial hypertension, diabetes, hypothalamicpituitary-adrenal axis suppression, obesity, cataracts, glaucoma, skin thinning leading to cutaneous striae and easy bruising, and muscle weakness. Other controller medications may act as steroid-sparing therapies because they have an advantage of reducing the corticosteroid intake.

Immunosuppressive drugs, such as azathioprine (Immurel, Azasan, Imuran), its metabolite 6-mercaptopurine (Purinethol), and methotrexate (Rheumatrex, Trexal) have shown to be effective in inducing response and remission.

The newest class of drug for treatment of Crohn's disease is biologics. Biologics are antibodies that target particular proteins and cells and then block the process that causes inflammation in the gut. These drugs include adalimumab (Humira®), certolizumab pegol (Cimzia®), infliximab (Remicade®), golimumab (Simponi®), natalizumab (Tysabri®) and vedolizumab (Entyvio®). Biologics interfere with the body's inflammatory response in Crohn's disease by targeting specific molecular players in the process such as cytokines—specialized proteins that play a role in increasing or decreasing inflammation. One such target is tumor necrosis factor (TNF) alpha, which is targeted by the TNF inhibitors of certolizumab pegol, adalimumab, and infliximab

Unfortunately, sometimes, these therapies are characterized by incomplete response and a substantial risk of adverse effects. TNF inhibitors and other biologics are still associated with safety concerns reported during treatment of Crohn's disease: injection site reactions, upper respiratory infections are the most commonly reported; tuberculosis and other opportunistic infections are rare, demyelination, drug-induced lupus and possibly myeloma being extremely rare.

In a systematic review on the use of TNF alpha inhibitors for Crohn's disease, notably the registered drugs of adalimumab and infliximab, the authors concluded that the initial good response was generally not maintained with extended treatment (Dretzke J, Edlin R, Round J et al., Health Technology Assessment 2011; Vol. 15: No. 6).

Therefore, there is still a need for effective drugs in the treatment of Crohn's disease.

There is also a need for safer drugs in the treatment of Crohn's disease.

### SUMMARY OF THE INVENTION

The invention aims to solve the technical problem of providing an active ingredient for the treatment of Crohn's disease.

The invention also aims to solve the technical problem of providing an active ingredient for an efficient treatment of Crohn's disease, especially in human patients.

The invention aims to provide an efficient treatment for Crohn's disease at an appropriate dose, route of administration, and daily intake.

One particular aim of the invention is to provide a treatment for moderate to severe Crohn's disease.

Another aim of the invention is to provide an alternative treatment for patients intolerant or with unsatisfactory response to immunosuppressive drugs and/or TNF inhibitors.

Another aim of the invention is to provide an alternative treatment for patients who are naïve to biologics, including TNF inhibitors.

The invention also aims to solve the technical problem of providing an active ingredient that improves prior art methods for the treatment of moderate to severe Crohn's disease. One particular aim of the invention therefore is to provide an alternative treatment for patients for administration in association with immunosuppressive or biologic agents.

### DEFINITIONS

By “CDAI” is to be understood the Crohn's Disease Activity Index. It represents a disease specific index that evaluates the activity of Crohn's disease in several domains, each of which evaluating a specific aspect of the disease. The CDAI sums up the weighted value of each item of the domain and quantifies the global disease severity in a final numerical score. The CDAI represents a numerical estimation of the patient symptoms.

TABLE 1
Description of CDAI domains and weights

| Item | Weight |
|---|---|
| Number of liquid or very soft stools | 2 |
| Abdominal pain score in one week (rating: 0-3) | 5 |
| General well-being (rating: 1-4) | 7 |
| Sum of findings per week: | 20 |
| Arthritis or arthralgia | |
| Mucocutaneous lesions (e.g., erythema nodosum, | |
| aphthous ulcers, pyoderma gangrenosum) | |
| Iritis/uveitis | |
| Anal disease (abscess, fistula) | |
| Other bowel related fistula | |
| Febril episodes >37.7° during past week | |
| Antidiarrheal use (loperamide or opiates) | 30 |
| Abdominal mass (none = 0, equivocal = 2, present = 5) | 10 |
| 47 minus hematocrit (males) or 42 minus hematocrit (females) | 6 |
| 100 × (1 − [body weight divided by standard weight]) | 1 |

This assessment is performed at screening, baseline, weeks 2, 4 and 8, for example. The CDAI sums up the weighted value of each item of the domain and quantifies the global disease severity in a final numerical score according to Best W R, Becktel J M, Singleton J W Kern F Jr. Development of a Crohn's disease activity index. National Cooperative Crohn's Disease Study. Gastroenterology. 1976; 70(3):439-44.

Widely used benchmarks for CDAI are: CDAI<150 “clinical remission”, 150-219 “mild actively disease”, 220-450 “moderately active disease”, and above 450 “very severe disease” (Khanna R, et al. A retrospective analysis: the development of patient reported outcome measures for the assessment of Crohn's disease activity. Aliment Pharmacol Ther. 2015 January; 41(1):77-86).

“Active disease” may refer to CDAI values over 150. Individuals with mild-moderate disease (usually corresponding to a CDAI 150-219) are ambulatory and able to tolerate oral alimentation without manifestations of dehydration, systemic toxicity (high fevers, rigors, and prostration), abdominal tenderness, painful mass, intestinal obstruction, or >10% weight loss. Individuals who are considered to have moderate-severe disease (usually corresponding to a CDAI 220-450) are considered to have failed to respond to treatment for mild-moderate disease, or those with more prominent symptoms of fever, significant weight loss, abdominal pain or tenderness, intermittent nausea or vomiting (without obstructive findings), or significant anemia. Finally, those individuals who are considered to have severe disease (usually corresponding to a CDAI>450) are patients with persistent symptoms despite the introduction of conventional corticosteroids or biologic agents as outpatients, or individuals presenting with high fevers, persistent vomiting, evidence of intestinal obstruction, significant peritoneal signs such as involuntary guarding or rebound tenderness, cachexia, or evidence of an abscess (Lichtenstein G R, et al. Management of Crohn's disease in adults. Am J Gastroenterol. 2009 February; 104(2):465-83; quiz 464, 484). By “patient with unsatisfactory response to immunosuppressive drug and/or biologic agent (including TNF inhibitor)”, it may be understood that said patient has been previously treated by immunosuppressive drug and/or biologic agent and does not respond to the treatment or responds to the treatment with high and too toxic doses of said immunosuppressive drug and/or biologic agent.

In one embodiment, patients have a CDAI>220. In one embodiment, patients have a CDAI>250. In one embodiment, patients have a CDAI>275. In one embodiment, patients have a CDAI>300. In one embodiment, patients have a CDAI>325. In another embodiment, patients have a CDAI>350.

In one embodiment, patients have a CDAI comprised between 220 and 450 (220<CDAI≦450). In one embodiment, patients have a CDAI comprised between 250 and 450 (250<CDAI≦450). In one embodiment, patients have a CDAI comprised between 275 and 450 (275<CDAI≦450). In one embodiment, patients have a CDAI comprised between 300 and 450 (300<CDAI≦450). In one embodiment, patients have a CDAI comprised between 325 and 450 (325<CDAI≦450). In another embodiment, patients have a CDAI comprised between 350 and 450 (350<CDAI≦450).

In the present invention, patients with a CDAI superior to 350 (CDAI>350), preferably comprised between 350 and 450 (350<CDAI≦450), are considered to suffer from moderately-severe active Crohn's disease. In one embodiment, patients have a CDAI superior to 350 (CDAI>350), preferably comprised between 350 and 450 (350<CDAI≦450), and are refractory to treatments for Crohn's disease. In other words, in one embodiment, patients suffer from moderately-severe active Crohn's disease and are refractory to treatments for Crohn's disease.

In a particular embodiment, by “patient with unsatisfactory response to immunosuppressive drug and/or biologic agent” it may be understood a patient with active Crohn's disease, in particular with a CDAI superior to 150, preferably superior or equal to 220, after at least 3 months under stable dose of immunosuppressive drug and/or biologic agent.

In one embodiment, by “patient intolerant to immunosuppressive drug and/or biologic agent”, it may be understood a patient who has had to discontinue such treatment at any time for tolerability or safety reasons.

In a particular embodiment, by “patient naïve to biologics, including TNF inhibitors” it may be understood a patient with active Crohn's disease, in particular with a CDAI superior to 150, preferably superior or equal to 220, has never been treated with biologics or no previous exposure to TNF inhibitors.

In a particular embodiment, by “administration in association with immunosuppressive drug and/or biologic agent” it may be understood a patient with active Crohn's disease, in particular with a CDAI superior to 150, preferably superior or equal to 220, under stable dose of said immunosuppressive drug or biologic agent.

“Remission” may refer to patients who are asymptomatic or without inflammatory sequelae. Patients requiring steroids to maintain well-being may be considered to be steroid dependent and may be not considered to be in remission (see Practice Parameters Committee of the American College of Gastroenterology, Hanauer, 1997).

By “immunosuppressive drugs” it is meant drugs that reduce or suppress the immune response.

“Masitinib” designates also an acceptable salt thereof, especially masitinib mesilate, even not explicitly stated.

The terms “as defined according to the invention” refer to any embodiments or aspects of the invention alone or in combination without limitation, including any preferred embodiments and variants, including any embodiments and features relating to tyrosine kinase inhibitor, mast cell inhibitor or c-Kit inhibitor, preferably masitinib, the method of treatment of Crohn's disease, pharmaceutical compositions and any combination with other pharmaceutically active ingredient(s).

### DESCRIPTION OF THE INVENTION

The etiology of Crohn's disease is unknown, but suggested possibilities include genetic, environmental, immunologic, and infectious causes. There is also some association with diet, and the disease affects more smokers than expected.

Pathophysiologically, Crohn's disease involves an immune system dysfunction. An imbalance in local mucosal production of pro-inflammatory cytokines over anti-inflammatory cytokines is theorized to cause the well-demarcated, discontinuous, transmural, ulcerative lesions characteristic of the disease. The most abundant cytokine is interleukin-1 (IL-1). Other cytokines activated in the inflammatory process are IL-6, tumor necrosis factor-α (TNF-α), and the chemokine IL-8. Cytokines cause differentiation of lymphocytes to different types of T cells. These cytokines serve to stimulate the immune system and cause an inflammatory reaction, thus producing tissue damage in the intestinal mucosa.

Mast cells may also be involved in Crohn's disease by their ability to release said inflammatory cytokines, and in particular TNF-α, or degrading enzymes that have been shown to be involved in the inflammatory course of the disease (Nishida Y, et al. Hepatogastroenterology 2002; 49(45):678-82) and Shao-Heng He. World J Gastroenterol 2004; 10(3):309-318). Indeed, mast cells may be a key cell type actively involved in the pathogenesis of IBD, with mast cell increasing number and degranulation releasing inflammatory cytokine including histamine and tryptase. In turn, both histamine and tryptase stimulate adjacent mast cells in the gut or generate positive feedback to further stimulate the host mast cell, thereby, providing at least two pathways for mast cells to self-amplify their degranulation signals (Shao-Heng He. World J Gastroenterol 2004; 10(3):309-318).

The present invention thus relates to a method for the treatment of Crohn's disease wherein said method comprises administering to a mammal in need thereof, at least one tyrosine kinase inhibitor, mast cell inhibitor or c-Kit inhibitor.

In one embodiment, tyrosine kinase inhibitor, mast cell inhibitor or c-Kit inhibitor is administered to a human patient.

In one embodiment, said tyrosine kinase inhibitor, mast cell inhibitor or c-Kit inhibitor is administered in combination with at least one pharmaceutically active ingredient. Said pharmaceutically active ingredient is preferably active in the treatment of Crohn's disease. Such pharmaceutically active ingredient is preferably chosen among aminosalicylates, corticosteroids, immunosuppressive drugs, biologics and TNF inhibitors.

In one embodiment, said patient has a CDAI>220 at baseline, preferably comprised between 220 and 450 (220<CDAI≦450) at baseline. In one embodiment, said patient has a CDAI>250 at baseline, preferably comprised between 250 and 450 (250<CDAI≦450) at baseline. In one embodiment, said patient has a CDAI>275 at baseline, preferably comprised between 275 and 450 (275<CDAI≦450) at baseline. In one embodiment, said patient has a CDAI>300 at baseline, preferably comprised between 300 and 450 (300<CDAI≦450) at baseline. In one embodiment, said patient has a CDAI>325 at baseline, preferably comprised between 325 and 450 (325<CDAI≦450) at baseline. In another embodiment, said patient has a CDAI>350 at baseline, preferably comprised between 350 and 450 (350<CDAI≦450) at baseline.

In one embodiment, said patient suffers from moderately-severe active Crohn's disease with a CDAI superior to 350 (CDAI>350) at baseline, preferably comprised between 350 and 450 (350<CDAI≦450) at baseline. In one embodiment, said patient suffers from refractory moderately-severe active Crohn's disease with a CDAI superior to 350 (CDAI>350) at baseline, preferably comprised between 350 and 450 (350<CDAI≦450) at baseline.

In one embodiment, said patient is selected among one of the following groups of patients:
A group of patients who never received any therapy for Crohn's disease or have previously received controller medications selected from aminosalicylate or corticosteroid agents.
A group of patients who have previously received azathioprine, 6-mercaptopurine, methotrexate, or one prior course of an immunosuppressive drug.
A group of patients refractory to at least one previous course of immunosuppressive treatment for Crohn's disease.
A group of patients who have previously received adalimumab, certolizumab pegol, infliximab, golimumab, natalizumab, vedolizumab, or one prior course of a biologic and/or TNF alpha inhibitor.
A group of patients refractory to at least one previous course of biologic and/or TNF alpha inhibitor treatment for Crohn's disease.

In one embodiment, said patient has a previous and/or concomitant treatment with a corticosteroid, and/or an immunosuppressive drug. In one particular embodiment, “previous treatment with a corticosteroid” means a treatment with a stable daily dose of 25 mg prednisolone or equivalent, administered for a minimum of 2 weeks prior to treatment initiation with said tyrosine kinase inhibitor, mast cell inhibitor or c-Kit inhibitor.

In a particular embodiment, “previous treatment with an immunosuppressive drug” means a treatment with an immunosuppressive drug such as azathioprine, 6-mercaptopurine or methotrexate, administered for at least 3 months at stable dosage prior to treatment initiation with said tyrosine kinase inhibitor, mast cell inhibitor or c-Kit inhibitor. In one embodiment, said tyrosine kinase inhibitor, mast cell inhibitor or c-Kit inhibitor, preferably masitinib is administered as first, second or third-line treatment of Crohn's disease.

In a specific embodiment, the invention relates to a method wherein said tyrosine kinase inhibitor, mast cell inhibitor or c-Kit inhibitor, preferably masitinib, benefits patients refractory to treatments for Crohn's disease, more particularly refractory to immunosuppressive drugs and/or biologics, especially after failure to anti-TNF therapy. “Refractory to Crohn's disease” means here, that a previous treatment did not work as expected for the treatment of Crohn's disease; in other words, the patient was resistant, relapsed, or intolerant to the administered treatment.

Preferably, said tyrosine kinase inhibitor or mast cell inhibitor is an inhibitor of kinase activity selected from the tyrosine kinases of: c-Kit, PDGFR, Lyn and Fyn.

Preferably, said tyrosine kinase inhibitor, mast cell inhibitor or c-Kit inhibitor is masitinib or a pharmaceutically acceptable salt thereof, and even more preferably, a masitinib mesilate salt.

In one embodiment, said tyrosine kinase inhibitor, mast cell inhibitor or c-Kit inhibitor, preferably masitinib is administered at a daily dose of 1.0 to 10.0 mg/kg/day (mg per kg bodyweight per day).

In one embodiment, said tyrosine kinase inhibitor, mast cell inhibitor or c-Kit inhibitor, preferably masitinib is administered at a dose of 1.5, 3.0, 4.5, 6.0, 7.5, or 9.0 mg/kg/day, more preferably 3.0, 4.5 or 6 mg/kg/day.

In one embodiment, said tyrosine kinase inhibitor, mast cell inhibitor or c-Kit inhibitor, preferably masitinib, is initially administered at a dose of 3 mg/kg/day during 4 weeks, then 4.5 mg/kg/day during 4 weeks and then 6 mg/kg/day thereafter. Each switch is subjected to toxicity controls, including but not limited to: 4-week treatment period with the same dose of study treatment AND no suspected severe adverse event was reported AND no suspected adverse event led to treatment interruption AND no suspected adverse event is ongoing at the time of the dose increase, regardless of its severity.

In one embodiment, said tyrosine kinase inhibitor, mast cell inhibitor or c-Kit inhibitor, preferably masitinib is dose escalated by increments of 1.5 mg/kg/day to reach a maximum of 10.0 mg/kg/day.

In one embodiment, said tyrosine kinase inhibitor, mast cell inhibitor or c-Kit inhibitor, preferably masitinib is dose reduced by increments of 1.5 mg/kg/day to reach a minimum of 1.5 mg/kg/day.

Any dose indicated herein refers to the amount of active ingredient as such, not to its salt form.

Given that the masitinib dose in mg/kg/day used in the described dose regimens refers to the amount of active ingredient masitinib, compositional variations of a pharmaceutically acceptable salt of masitinib mesilate will not change the said dose regimens.

In one embodiment, said tyrosine kinase inhibitor, mast cell inhibitor or c-Kit inhibitor, preferably masitinib is administered orally.

In one embodiment, said tyrosine kinase inhibitor, mast cell inhibitor or c-Kit inhibitor, preferably masitinib is administered once or twice a day.

In one embodiment, said tyrosine kinase inhibitor, mast cell inhibitor or c-Kit inhibitor is administered in combination with said at least one pharmaceutically active ingredient preferably chosen among aminosalicylates, corticosteroids, immunosuppressive drugs, biologics and TNF inhibitors, more preferably immunosuppressive drugs.

In one embodiment, aminosalicylates are chosen among the group consisting of balsalazide (Colazal), olsalazine (DiPentum), mesalamine (Asacol and Pentasa), and sulfasalazine (Azulfidine). In one embodiment, corticosteroids are chosen among the group consisting of budesonide, prednisolone and dexamethasone. In one embodiment, immunosuppressive drugs are chosen among the group consisting of azathioprine, 6-mercaptopurine and methotrexate. In one embodiment TNF inhibitors are TNF alpha inhibitors, for example adalimumab (Humira®), certolizumab pegol (Cimzia®), infliximab (Remicade®), golimumab (Simponi®), natalizumab (Tysabri®) and vedolizumab (Entyvio®).

In a particular embodiment, said tyrosine kinase inhibitor, mast cell inhibitor or c-Kit inhibitor is administered in combination with immunosuppressive drugs, preferably azathioprine, 6-mercaptopurine and methotrexate.

In one embodiment, said tyrosine kinase inhibitor, mast cell inhibitor or c-Kit inhibitor is administered in combination with said at least one pharmaceutically active ingredient in a combined preparation for simultaneous, separate, or sequential use.

In one embodiment, said tyrosine kinase inhibitor, mast cell inhibitor or c-Kit inhibitor is useful in the treatment of patients with moderate to severe Crohn's disease.

In a particular embodiment, said tyrosine kinase inhibitor, mast cell inhibitor or c-Kit inhibitor is useful in the treatment of patients intolerant or with unsatisfactory response to immunosuppressive drug and/or biologic agent (including TNF-inhibitor). In particular, said tyrosine kinase inhibitor, mast cell inhibitor or c-Kit inhibitor is useful in the treatment of patients intolerant or with unsatisfactory response to immunosuppressive drugs chosen among the group consisting of azathioprine, 6-mercaptopurine and methotrexate and/or to TNF-inhibitors chosen among adalimumab, certolizumab pegol, infliximab, golimumab, natalizumab or vedolizumab.

In one embodiment, the method of the invention for the treatment of patients suffering from Crohn's disease including patients previously treated with anti-TNF therapy comprises meaningful improvement in CDAI responses and/or associated inflammation.

In a particular embodiment, said tyrosine kinase inhibitor, mast cell inhibitor or c-Kit inhibitor induces a clinical remission, represented by a CDAI<150 and an absence of surgery, or a clinical response represented by a decrease in CDAI≧70 or an enhanced clinical response, defined by a decrease in CDAI≧100.

In one embodiment, said tyrosine kinase inhibitor, mast cell inhibitor or c-Kit inhibitor reduces inflammation in patients with Crohn's disease, in particular reduces the CRP level for example by 10% to 50%, preferably by 20% to 40% from its baseline.

The invention also relates to a tyrosine kinase inhibitor, mast cell inhibitor or c-Kit inhibitor, preferably masitinib, as defined according to the present invention, for use in a treatment of Crohn's disease.

The invention also relates to a tyrosine kinase inhibitor, mast cell inhibitor or c-Kit inhibitor, preferably masitinib, as defined according to the present invention, for use in a treatment of Crohn's disease, in combination with at least one pharmaceutically active ingredient, preferably immunosuppressive drugs such as azathioprine, 6-mercaptopurine and methotrexate.

The invention also relates to a pharmaceutical composition or kit comprising a tyrosine kinase inhibitor, mast cell inhibitor or c-Kit inhibitor, preferably masitinib, for use in a method for the treatment of Crohn's disease as defined according to the present invention.

The invention also relates to a pharmaceutical composition or kit comprising a tyrosine kinase inhibitor, mast cell inhibitor or c-Kit inhibitor, preferably masitinib, and at least one other pharmaceutically active ingredient, preferably chosen among aminosalicylates, corticosteroids, immunosuppressive drugs, biologics and TNF inhibitors, more preferably immunosuppressive drugs.

The invention also relates to the use of a tyrosine kinase inhibitor, mast cell inhibitor or c-Kit inhibitor, preferably masitinib, for the preparation of a medicament, or a pharmaceutical composition, for the treatment of Crohn's disease, optionally in combination with at least one other pharmaceutically active ingredient, preferably chosen among aminosalicylates, corticosteroids, immunosuppressive drugs, biologics and TNF inhibitors, more preferably immunosuppressive drugs.

The tyrosine kinase inhibitor, mast cell inhibitor or c-Kit inhibitor, and the optional at least one pharmaceutically active ingredient, are administered in a dosage regimen that comprises a therapeutically effective amount.

Tyrosine kinases are receptor type or non-receptor type proteins, which transfer the terminal phosphate of ATP to tyrosine residues of proteins thereby activating or inactivating signal transduction pathways. These proteins are known to be involved in many cellular mechanisms, which in case of disruption, lead to disorders such as abnormal cell proliferation and migration as well as inflammation. A tyrosine kinase inhibitor is a drug that inhibits tyrosine kinases, thereby interfering with signaling processes within cells. Blocking such processes can stop the cell growing and dividing.

In one embodiment, the tyrosine kinase inhibitor of the invention has the following formula [A]:

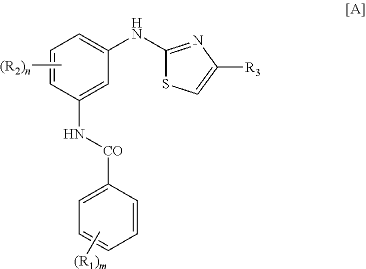

wherein R₁ and R₂, are selected independently from hydrogen, halogen, a linear or branched alkyl, cycloalkyl group containing from 1 to 10 carbon atoms, trifluoromethyl, alkoxy, cyano, dialkylamino, and a solubilizing group,
m is 0-5 and n is 0-4;
the group R₃ is one of the following:
(i) an aryl group such as phenyl or a substituted variant thereof bearing any combination, at any one ring position, of one or more substituents such as halogen, alkyl groups containing from 1 to 10 carbon atoms, trifluoromethyl, cyano and alkoxy;
(ii) a heteroaryl group such as 2, 3, or 4-pyridyl group, which may additionally bear any combination of one or more substituents such as halogen, alkyl groups containing from 1 to 10 carbon atoms, trifluoromethyl and alkoxy;
(iii) a five-membered ring aromatic heterocyclic group such as for example 2-thienyl, 3-thienyl, 2-thiazolyl, 4-thiazolyl, 5-thiazolyl, which may additionally bear any combination of one or more substituents such as halogen, an alkyl group containing from 1 to 10 carbon atoms, trifluoromethyl, and alkoxy;
or a pharmaceutically acceptable salt or solvate thereof.

Tyrosine kinase inhibitors of formula [A] can preferably be used as c-Kit inhibitors.

Unless otherwise specified, the below terms used herein are defined as follows: As used herein, the term an “aryl group” means a monocyclic or polycyclic-aromatic radical comprising carbon and hydrogen atoms. Examples of suitable aryl groups include, but are not limited to, phenyl, tolyl, anthracenyl, fluorenyl, indenyl, azulenyl, and naphthyl, as well as benzo-fused carbocyclic moieties such as 5,6,7,8-tetrahydronaphthyl. An aryl group can be unsubstituted or substituted with one or more substituents. In one embodiment, the aryl group is a monocyclic ring, wherein the ring comprises 6 carbon atoms, referred to herein as “(C6)aryl”.

As used herein, the term “alkyl group” means a saturated straight chain or branched non-cyclic hydrocarbon having from 1 to 10 carbon atoms. Representative saturated straight chain alkyls include methyl, ethyl, n-propyl, n-butyl, n-pentyl, n-hexyl, n-heptyl, n-octyl, n-nonyl and n-decyl; while saturated branched alkyls include isopropyl, sec-butyl, isobutyl, tert-butyl, isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, 2,3-dimethylpentyl, 2,4-dimethylpentyl, 2,3-dimethylhexyl, 2,4-dimethylhexyl, 2,5-dimethylhexyl, 2,2-dimethylpentyl, 2,2-dimethylhexyl, 3,3-dimethylpentyl, 3,3-dimethylhexyl, 4,4-dimethylhexyl, 2-ethylpentyl, 3-ethylpentyl, 2-ethylhexyl, 3-ethylhexyl, 4-ethylhexyl, 2-methyl-2-ethylpentyl, 2-methyl-3-ethylpentyl, 2-methyl-4-ethylpentyl, 2-methyl-2-ethylhexyl, 2-methyl-3-ethylhexyl, 2-methyl-4-ethylhexyl, 2,2-diethylpentyl, 3,3-diethylhexyl, 2,2-diethylhexyl, 3,3-diethylhexyl and the like. Alkyl groups included in compounds of this invention may be optionally substituted with one or more substituents.

As used herein, the term “alkoxy” refers to an alkyl group which is attached to another moiety by an oxygen atom. Examples of alkoxy groups include methoxy, isopropoxy, ethoxy, tert-butoxy, and the like. Alkoxy groups may be optionally substituted with one or more substituents.

As used herein, the term “heteroaryl” or like terms means a monocyclic or polycyclic heteroaromatic ring comprising carbon atom ring members and one or more heteroatom ring members (such as, for example, oxygen, sulfur or nitrogen). Typically, a heteroaryl group has from 1 to about 5 heteroatom ring members and from 1 to about 14 carbon atom ring members. Representative heteroaryl groups include pyridyl, 1-oxo-pyridyl, furanyl, benzo[1,3]dioxolyl, benzo[1,4]dioxinyl, thienyl, pyrrolyl, oxazolyl, imidazolyl, thiazolyl, isoxazolyl, quinolinyl, pyrazolyl, isothiazolyl, pyridazinyl, pyrimidinyl, pyrazinyl, triazinyl, triazolyl, thiadiazolyl, isoquinolinyl, indazolyl, benzoxazolyl, benzofuryl, indolizinyl, imidazopyridyl, tetrazolyl, benzimidazolyl, benzothiazolyl, benzothiadiazolyl, benzoxadiazolyl, indolyl, tetrahydroindolyl, azaindolyl, imidazopyridyl, quinazolinyl, purinyl, pyrrolo[2,3]pyrimidinyl, pyrazolo[3,4]pyrimidinyl, imidazo[1,2-a]pyridyl, and benzo(b)thienyl. A heteroatom may be substituted with a protecting group known to those of ordinary skill in the art, for example, the hydrogen on a nitrogen may be substituted with a tert-butoxycarbonyl group. Heteroaryl groups may be optionally substituted with one or more substituents. In addition, nitrogen or sulfur heteroatom ring members may be oxidized. In one embodiment, the heteroaromatic ring is selected from 5-8 membered monocyclic heteroaryl rings. The point of attachment of a heteroaromatic or heteroaryl ring to another group may be at either a carbon atom or a heteroatom of the heteroaromatic or heteroaryl rings.

The term “heterocycle” as used herein, refers collectively to heterocycloalkyl groups and heteroaryl groups.

As used herein, the term “heterocycloalkyl” means a monocyclic or polycyclic group having at least one heteroatom selected from O, N or S, and which has 2-11 carbon atoms, which may be saturated or unsaturated, but is not aromatic. Examples of heterocycloalkyl groups include (but are not limited to): piperidinyl, piperazinyl, 2-oxopiperazinyl, 2-oxopiperidinyl, 2-oxopyrrolidinyl, 4-piperidonyl, pyrrolidinyl, hydantoinyl, valerolactamyl, oxiranyl, oxetanyl, tetrahydropyranyl, tetrahydrothiopyranyl, tetrahydropyrindinyl, tetrahydropyrimidinyl, tetrahydrothiopyranyl sulfone, tetrahydrothiopyranyl sulfoxide, morpholinyl, thiomorpholinyl, thiomorpholinyl sulfoxide, thiomorpholinyl sulfone, 1,3-dioxolane, tetrahydrofuranyl, dihydrofuranyl-2-one, tetrahydrothienyl, and tetrahydro-1,1-dioxothienyl. Typically, monocyclic heterocycloalkyl groups have 3 to 7 members.

Preferred 3 to 7 membered monocyclic heterocycloalkyl groups are those having 5 or 6 ring atoms. A heteroatom may be substituted with a protecting group known to those of ordinary skill in the art, for example, the hydrogen on a nitrogen may be substituted with a tert-butoxycarbonyl group. Furthermore, heterocycloalkyl groups may be optionally substituted with one or more substituents. In addition, the point of attachment of a heterocyclic ring to another group may be at either a carbon atom or a heteroatom of a heterocyclic ring. Only stable isomers of such substituted heterocyclic groups are contemplated in this definition.

As used herein the term “substituent” or “substituted” means that a hydrogen radical on a compound or group is replaced with any desired group that is substantially stable to reaction conditions in an unprotected form or when protected using a protecting group.

Examples of preferred substituents are those found in the exemplary compounds and embodiments disclosed herein, as well as halogen (chloro, iodo, bromo, or fluoro); alkyl; alkenyl; alkynyl; hydroxy; alkoxy; nitro; thiol; thioether; imine; cyano; amido; phosphonato; phosphine; carboxyl; thiocarbonyl; sulfonyl; sulfonamide; ketone; aldehyde; ester; oxygen (—O); haloalkyl (e.g., trifluoromethyl); cycloalkyl, which may be monocyclic or fused or non-fused polycyclic (e.g., cyclopropyl, cyclobutyl, cyclopentyl, or cyclohexyl), or a heterocycloalkyl, which may be monocyclic or fused or non-fused polycyclic (e.g., pyrrolidinyl, piperidinyl, piperazinyl, morpholinyl, or thiazinyl), monocyclic or fused or non-fused polycyclic aryl or heteroaryl (e.g., phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridyl, quinolinyl, isoquinolinyl, acridinyl, pyrazinyl, pyridazinyl, pyrimidinyl, benzimidazolyl, benzothiophenyl, or benzofuranyl); amino (primary, secondary, or tertiary); CO₂CH₃; CONH₂; OCH₂CONH₂; NH₂; SO₂NH₂; OCHF₂; CF₃; OCF₃; and such moieties may also be optionally substituted by a fused-ring structure or bridge, for example —OCH₂O—. These substituents may optionally be further substituted with a substituent selected from such groups. In certain embodiments, the term “substituent” or the adjective “substituted” refers to a substituent selected from the group consisting of an alkyl, an alkenyl, an alkynyl, an cycloalkyl, an cycloalkenyl, a heterocycloalkyl, an aryl, a heteroaryl, an aralkyl, a heteraralkyl, a haloalkyl, —C(O)NR₁₁R₁₂, —NR₁₃C(O)R₁₄, a halo, —OR₁₃, cyano, nitro, a haloalkoxy, —C(O)R₁₃, —NR₁₁R₁₂, —SR₁₃, —C(O)OR₁₃, —OC(O)R₁₃, —NR₁₃C(O)NR₁₁R₁₂, —OC(O)NR₁₁R₁₂, —NR₁₃C(O)OR₁₄, —S(O)ᵣR₁₃, —NR₁₃S(O)ᵣR₁₄, —OS(O)ᵣR₁₄, S(O)ᵣNR₁₁R₁₂, —O, —S, and —N—R₁₃, wherein r is 1 or 2; R₁₁ and R₁₂, for each occurrence are, independently, H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl; or R₁₁ and R₁₂ taken together with the nitrogen to which they are attached is optionally substituted heterocycloalkyl or optionally substituted heteroaryl; and R₁₃ and R₁₄ for each occurrence are, independently, H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl.

In certain embodiments, the term “substituent” or the adjective “substituted” refers to a solubilizing group.

The term “solubilizing group” means any group which can be substantially ionized and that enables the compound to be soluble in a desired solvent, such as, for example, water or water-containing solvent. Furthermore, the solubilizing group can be one that increases the compound or complex's lipophilicity. Typically, the solubilizing group is selected from alkyl group substituted with one or more heteroatoms such as N, O, S, each optionally substituted with alkyl group substituted independently with alkoxy, amino, alkylamino, dialkylamino, carboxyl, cyano, or substituted with cycloheteroalkyl or heteroaryl, or a phosphate, or a sulfate, or a carboxylic acid. For example, by “solubilizing group” it is referred herein to one of the following:
an alkyl, cycloalkyl, aryl, heretoaryl group comprising either at least one nitrogen or oxygen heteroatom or which group is substituted by at least one amino group or oxo group;
an amino group which may be a saturated cyclic amino group which may be substituted by a group consisting of alkyl, alkoxycarbonyl, halogen, haloalkyl, hydroxyalkyl, amino, monoalkylamino, dialkylamino, carbamoyl, monoalkylcarbamoyl and dialkylcarbamoyl;
one of the structures a) to i) shown below, wherein the wavy line and the arrow line correspond to the point of attachment to core structure of Formula [A]:

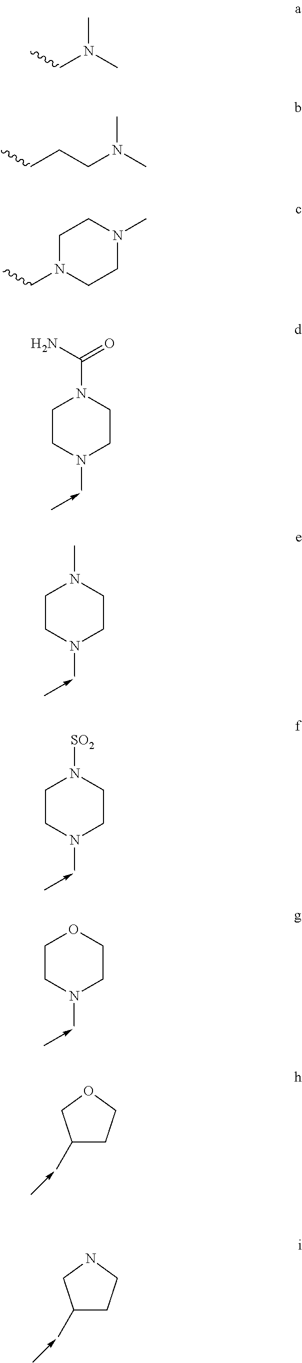

The term “cycloalkyl” means a saturated cyclic alkyl radical having from 3 to 10 carbon atoms. Representative cycloalkyls include cyclopropyl, 1-methylcyclopropyl, cyclobutyl, cyclopentyl, cyclohexyl, cycloheptyl, cyclooctyl, cyclononyl, and cyclodecyl. Cycloalkyl groups can be optionally substituted with one or more substituents.

The term “halogen” means —F, —Cl, —Br or —I.

In a particular embodiment the tyrosine kinase inhibitor of the invention has general formula [B],

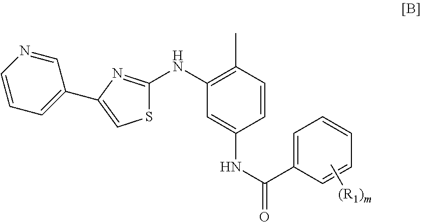

wherein:
R₁ is selected independently from hydrogen, halogen, a linear or branched alkyl, cycloalkyl group containing from 1 to 10 carbon atoms, trifluoromethyl, alkoxy, amino, alkylamino, dialkylamino, solubilizing group, and m is 0-5.

In one embodiment, the tyrosine kinase inhibitor, mast cell inhibitor or c-Kit inhibitor is masitinib or a pharmaceutically acceptable salt thereof, more preferably masitinib mesilate.

Masitinib is a c-Kit/PDGFR inhibitor with a potent anti-mast cell action.

New potent and selective c-Kit, platelet derived growth factor receptor (PDGFR) inhibitors are 2-(3-aminoaryl)amino-4-aryl-thiazoles described in AB Science's PCT application WO 2004/014903.

Masitinib is a small molecule drug, selectively inhibiting specific tyrosine kinases such as c-Kit, PDGFR, Lyn, and Fyn without inhibiting, at therapeutic doses, kinases associated with known toxicities (i.e. those tyrosine kinases or tyrosine kinase receptors attributed to possible tyrosine kinase inhibitor cardiac toxicity, including ABL, KDR and Src) [Dubreuil et al., 2009, PLoS ONE 2009. 4(9):e7258] [Davis et al., Nat Biotechnol 2011, 29(11): 1046-51]. The chemical name for masitinib is 4-(4-methylpiperazin-1-ylmethyl)-N-[4-methyl-3-(4-pyri din-3ylthiazol-2-ylamino) phenyl]benzamide—CAS number 790299-79-5, and the structure is shown below. Masitinib was first described in U.S. Pat. No. 7,423,055 and EP1525200B1. A detailed procedure for the synthesis of masitinib mesilate is given in WO2008/098949.

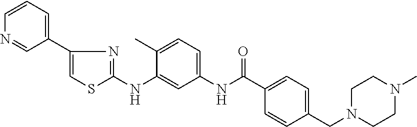 [SMILES: Cc1ccc(NC(=O)c2ccc(CN3CCN(C)CC3)cc2)cc1Nc1nc(-c2cccnc2)cs1]

Masitinib's main kinase target is c-Kit, for which it has been shown to exert a strong inhibitory effect on wild-type and juxtamembrane-mutated c-Kit receptors, resulting in cell cycle arrest and apoptosis of cell lines dependent on c-Kit signaling [Dubreuil et al., 2009, PLoS ONE, 4(9):e7258]. In vitro, masitinib demonstrated high activity and selectivity against c-Kit, inhibiting recombinant human wild-type c-Kit with an half inhibitory concentration (IC50) of 200±40 nM and blocking stem cell factor-induced proliferation and c-Kit tyrosine phosphorylation with an IC50 of 150±80 nM in Ba/F3 cells expressing human or mouse wild-type c-Kit. In addition to its anti-proliferative properties, masitinib can also regulate the activation of mast cells through its targeting of Lyn and Fyn, key components of the transduction pathway leading to IgE induced degranulation [Gilfillan et al., 2006, Nat Rev Immunol, 6:218-230] [Gilfillan et al., 2009, Immunological Reviews, 228:149-169]. This can be observed in the inhibition of FcεRI-mediated degranulation of human cord blood mast cells [Dubreuil et al., 2009, PLoS ONE; 4(9):e7258]. Masitinib is also an inhibitor of PDGFR α and β receptors. Recombinant assays show that masitinib inhibits the in vitro protein kinase activity of PDGFR-α and β with IC50 values of 540±60 nM and 800±120 nM. In Ba/F3 cells expressing PDGFR-α, masitinib inhibited PDGF-BB-stimulated proliferation and PDGFR-α tyrosine phosphorylation with an IC50 of 300±5 nM.

The present invention relates to a method for the treatment of Crohn's disease in a mammal, and especially a human patient, wherein said method comprises administering to a human patient in need thereof, a tyrosine kinase inhibitor, mast cell inhibitor or c-Kit inhibitor, especially masitinib or a pharmaceutically acceptable salt thereof, optionally combined with at least one pharmaceutically active ingredient.

In relation to the present invention, the term “treatment” (and its various grammatical forms) refers to preventing, curing, reversing, attenuating, alleviating, minimizing, suppressing or halting the deleterious effects of a disease state, disease progression, disease causative agent (e.g., bacteria or viruses) or other abnormal condition. For example, treatment may involve alleviating a symptom (i.e., not necessary all symptoms) of a disease or attenuating the progression of a disease.

Advantageously, the use or method comprises a long term administration of an effective amount of said tyrosine kinase inhibitor, mast cell inhibitor or c-Kit inhibitor, especially masitinib or a pharmaceutically acceptable salt thereof, over more than 3 months, preferably more than 6 months.

As is known to the person skilled in the art, various forms of excipients can be used adapted to the mode of administration and some of them can promote the effectiveness of the active molecule, e.g. by promoting a release profile rendering this active molecule overall more effective for the treatment desired.

The pharmaceutical compositions of the invention are thus able to be administered in various forms, more specially for example in an injectable, pulverizable or ingestible form, for example via the intramuscular, intravenous, subcutaneous, intradermal, oral, topical, rectal, vaginal, ophthalmic, nasal, transdermal or parenteral route. A preferred route is oral administration. The present invention notably covers the use of a compound according to the present invention for the manufacture of pharmaceutical composition.

Such medicament can take the form of a pharmaceutical composition adapted for oral administration, which can be formulated using pharmaceutically acceptable carriers well known in the art in suitable dosages. Such carriers enable the pharmaceutical compositions to be formulated as tablets, pills, dragees, capsules, liquids, gels, syrups, slurries, suspensions, and the like, for ingestion by the patient. In addition to the active ingredients, these pharmaceutical compositions may contain suitable pharmaceutically-acceptable carriers comprising excipients and auxiliaries which facilitate processing of the active compounds into preparations which can be used pharmaceutically. Further details on techniques for formulation and administration may be found in the latest edition of Remington's Pharmaceutical Sciences (Maack Publishing Co., Easton, Pa.).

According to a particular embodiment, the composition of the invention is an oral composition.

In one embodiment, compositions according to the invention may be in the form of tablets.

In one embodiment, composition according to the invention may comprise from 50 to 500 mg of said tyrosine kinase inhibitor, mast cell inhibitor or c-Kit inhibitor, especially masitinib or a pharmaceutically acceptable salt thereof. More particularly, the composition may comprise from 100 to 500 mg of said tyrosine kinase inhibitor, mast cell inhibitor or c-Kit inhibitor, especially masitinib or a pharmaceutically acceptable salt thereof, for example, 100, 200, 300, 400, or 500 mg.

The present invention is further illustrated by means of the following examples.

The data presented in these examples, and also in parts of the patent Description, are in part taken from preliminary analysis and as such represent a close approximation to the final, validated dataset.

### EXAMPLES

### Example 1

### Phase IIa—Clinical Trial

### Study Design

The objective of the phase IIa study was to evaluate, comparatively to a placebo, the activity of oral masitinib, administered at two dose levels during 8 weeks to patients with moderate to severe active Crohn's disease.

The study was a multicenter, double blind, randomized, placebo-controlled, parallel-group study. Patients were randomized to receive 4.5 or 6 mg/kg/day oral masitinib or placebo, in a 5:5:3 ratio, respectively. Patients were to be treated for 2 months with an option for treatment extension for patients showing significant improvement with manageable toxicity.

### Randomization

Patients were randomly allocated to one of the 3 following groups:
Masitinib, 4.5 mg/kg/day: total daily dose to be administered in two daily intakes (b.i.d.), morning and evening, during meals;
Masitinib, 6 mg/kg/day: total daily dose to be taken b.i.d., morning and evening, during meals;
Placebo: placebo tablets morning and evening, during meals.

Masitinib was supplied as 100 and 200 mg tablets of masitinib base, packaged in polyethylene bottles.

Inactive ingredients are microcrystalline cellulose, povidone, crospovidone, magnesium stearate, and coating agent, opadry orange.

Masitinib was to be taken twice daily during meals as indicated in the Table 2 below:

TABLE 2
Masitinib dosage

| Patient's | 1.5 mg/kg | | 3 mg/kg | | 4.5 mg/kg | | 6 mg/kg | |
|---|---|---|---|---|---|---|---|---|
| weight (kg) | am | pm | am | pm | am | pm | am | pm |
| <50 | Stop | | 100 | | 200 | | 200 | |
| [50-70] | 100 | | 200 | | 200 | 100 | 200 | 200 |
| ]70-90[ | 100 | | 200 | | 200 | 200 | 200 | 200 + |
| | | | | | | | | 100 |
| ≧90 | 200 | | 200 | 100 | 200 | 200 + | 200 + | 200 + |
| | | | | | | 100 | 100 | 100 |

Placebo tablets were identical to masitinib tablets without the active ingredient, placebo-coated tablets contained microcrystalline cellulose, povidone, crospovidone, magnesium stearate, and a coating agent (opadry orange), and were packaged in polyethylene bottles.

### Duration of Treatment

Patients were to receive treatment for up to 8 weeks. Patients were withdrawn during the first 8 weeks in the event of patient request, an adverse event considered intolerable by the patient or incompatible with continuation of the study according to the investigator, protocol violation or a worsening of the patient's disease status (i.e. an increase in CDAI score greater than 100 units in comparison with his/her baseline value).

Treatment could be continued for patients showing significant improvement. In this case, the follow-up of patients was to be identical to that of present protocol for week 4, i.e. every four weeks with same assessment during 6 months and after the extension visits were to be done every 3 months.

Extended treatment was to be continued until treatment escape, product registration or development cessation, whichever occurred first.

### Concomitant Treatments

Sixteen (70%) patients from the masitinib treatment group were receiving at least one treatment (i.e. immunotherapy or biologics) for moderate to severe Crohn's disease at study entry. The remaining two patients from the masitinib treatment group were treatment naïve (i.e. no previous treatment with immunotherapy or biologics).

Patients with Crohn's disease are commonly treated with a combination of corticosteroids and immunosuppressive agents, both of these agents contributing to lower the inflammation process.

Patients were categorized according to the previous treatment received, and classified as first-line (L1) if they never received any therapy for Crohn's disease or if they already received anti-inflammatory drugs such as aminosalicylate or corticosteroids, second-line (L2) if they already received azathioprine (Immurel) or one prior course of immunological therapy, and third-line (L3) if they already received biological agent.

TABLE 3
Number of patients by line of Crohn's disease
treatment, ITT population

| | All | Masitinib | Placebo |
|---|---|---|---|
| Treatment-line | (N = 23) | (N = 18) | (N = 5) |
| L1 | 4 (17.4%) | 2 (11.1%) | 2 (40.0%) |
| L2 | 2 (8.7%) | 2 (11.1%) | 0 (0.0%) |
| L3 | 17 (73.9%) | 14 (77.8%) | 3 (60.0%) |

Most of the patients (74%) received previous biological therapy for the treatment of Crohn's disease.

### Main Inclusion Criteria

1. Male or female patient aged 18 to 75 years, weight>40 kg, BMI>18.
2. Patient with at least a 6-month history of Crohn's disease from diagnosis.
3. Patient with Crohn's disease based on clinical history and radiologic or endoscopic findings within the previous 24 months. Patients with active disease following surgical resection must have radiological or endoscopic confirmation of Crohn's disease post-surgery.
4. Patients must have a 220≦CDAI (Crohn's disease activity index) score≦450 at baseline.

### Main Exclusion Criteria

1. Patients with known active or draining fistula.
2. Patients who do not meet any of the following criteria regarding concomitant medications for Crohn's disease:
   Any concomitant antibiotic must be stopped 2 weeks before treatment initiation.
   Concomitant use of oral and rectal 5-ASA compounds or rectal steroids is not permitted; these agents must have been discontinued at least 1 week prior to treatment initiation.
   Any concomitant oral steroids must not exceed a daily dose of 25 mg prednisolone or equivalent and must have been administered for a minimum of 2 weeks prior to treatment initiation and must have been at a stable dose for the last 2 weeks prior to treatment initiation. If recently discontinued, this product must have stopped at least 4 weeks prior to treatment initiation.
   Any concomitant treatment with azathioprine, 6-mercaptopurine, methotrexate must have been administered for at least 3 months at stable dosage prior to treatment initiation. If recently discontinued, this product must have been stopped at least 4 weeks prior to treatment initiation.
   Concomitant use of any other immunosuppressant (e.g., tacrolimus, cyclosporine, mycophenolate mofetil, thalidomide) is not permitted; these agents must have been discontinued at least 4 weeks prior to treatment initiation.
   Concomitant use of any anti-tumor necrosis factor (anti-TNF) therapy is not permitted; these agents must have been discontinued at least 8 weeks prior to treatment initiation.
   Concomitant use of herbal preparations.
   Concomitant use of any experimental agent is not permitted. These agents must have been discontinued at least 4 weeks prior to treatment initiation, or for a period equivalent to 5 half-lives of the agent (whichever is the longer).

### Endpoints

The primary endpoint was change from baseline in the Crohn's Disease Activity Index (CDAI). Secondary endpoints included:
C Reactive Protein (CRP) level (CRP level is raised in inflammatory bowel disease as Crohn's disease)
Fecal calprotectin level (this is a biochemical test for inflammatory bowel disease, which may replace the need for invasive colonoscopy. Levels are increased in patients with IBD and might be a predictive marker of relapse in Crohn's disease)

### Study Results

A total of 23 patients were randomized, 18 to masitinib (8 at 4.5 mg/kg/day and 10 at 6 mg/kg/day) and 5 to placebo. Of the patients allocated to the 4.5 mg/kg/day group, 4 were treated at 3 mg/kg/day, 2 were initially treated at 3 mg/kg/day and increased their dosage to 4.5 mg/kg/day after 4 weeks of treatment, and 2 were treated at 4.5 mg/kg/day. A total of 21 patients (16 masitinib, 5 placebo) were considered to be the per protocol.

Fifteen patients (65.2%) completed the planned initial 8-week treatment period, eight (34.8%) of whom continued in the extension period under blinded treatment (2 placebo, 6 masitinib).

Datasets presented hare include the ‘observed cases’ (OC) dataset with no replacement of missing data, and ‘last observed case carried forward’ (LOCF) dataset.

The ITT population (Intent-To-Treat population) consisted of all randomized patients whether they had received study drug or not, and included all 23 patients.

The per-protocol population (PP population) consisted of 21 patients. Two patients, for which there was no post-baseline CDAI assessment, were excluded from PP due to insufficient exposure duration.

### CDAI Response Rate

Table 4 shows clinical response at weeks 4 and 8 in the ITT population.

A clinical response was reported in 8 of 16 (50%) OC masitinib patients at week 4, compared to one (20%) responder under placebo.

A high rate of clinical responses occurred at week 8 in the OC masitinib group, with a clinical response reported in 7 of 10 (70%) masitinib patients, compared to 1 responder (20%) under placebo. Of the masitinib responders at week 8, five (50%) had enhanced responses from week 4. Results were repeated in the LOCF dataset.

TABLE 4
Number of patients with CDAI response at week 8 (ITT population)

| | Week 4 | | Week 8 | |
|---|---|---|---|---|
| | Masitinib | Placebo | Masitinib | Placebo |
| Observed Cases | | | | |
| N | 16 | 5 | 10 | 5 |
| CDAI response | 8 (50.0%) | 1 (20.0%) | 7 (70.0%) | 1 (20.0%) |
| 95% CI | [28.0%; 72.0%] | [3.6%; 62.4%] | [39.7%; 89.2%] | [3.6%; 62.4%] |
| P-value | 0.338 (F) | | 0.119 (F) | |
| CDAI enhanced response | 5 (31.3%) | 1 (20.0%) | 5 (50.0%) | 1 (20.0%) |
| 95% CI | [14.2%; 55.6%] | [3.6%; 62.4%] | [23.7%; 76.3%] | [3.6%; 62.4%] |
| P-value | 1.000 (F) | | 0.580 (F) | |
| LOCF | | | | |
| N | 16 | 5 | 16 | 5 |
| CDAI response | 8 (50.0%) | 1 (20.0%) | 7 (43.8%) | 1 (20.0%) |
| 95% CI | [28.0%; 72.0%] | [3.6%; 62.4%] | [23.1%; 66.8%] | [3.6%; 62.4%] |
| P-value | 0.338 (F) | | 0.606 (F) | |
| CDAI enhanced response | 5 (31.3%) | 1 (20.0%) | 5 (31.3%) | 1 (20.0%) |
| 95% CI | [14.2%; 55.6%] | [3.6%; 62.4%] | [14.2%; 55.6%] | [3.6%; 62.4%] |
| P-value | 1.000 (F) | | 1.000 (F) | |

(F) Fisher's exact test;
*Response: “at least 70 pts decrease”;
**Enhanced response: “at least 100 pts decrease”.

Analysis showed that masitinib group had a higher rate of clinical response than did the placebo group at weeks 4 and 8 (50% versus 20%, and 70% versus 20%, respectively).

From the 7/10 masitinib patients presenting a clinical response at week 8, 5/10 (50%) had an enhanced response. Complete remission was observed in two masitinib treated patients during the first 8-week period; after which one additional remission occurred.

CDAI Absolute and Relative Change from Baseline

For CDAI change relative to baseline, a negative change (i.e. a decrease in overall score) indicates improvement in disease activity.

CDAI absolute and relative changes from baseline are presented in Table 5.

In the observed cases ITT population, the mean change in CDAI for placebo patients was a decrease of 13.2 units (range [−153; −59]) at week 8 compared to a decrease of −86 (range [−282; −163]) for masitinib-treated patients. This represents a 23% improvement of mean disease activity in patients treated with masitinib, compared with only 4.6% improvement in placebo patients.

The onset of the treatment effect was rapid, evident as early as 4 weeks after the initiation of treatment (with 8 clinical responses and a mean CDAI decrease of 80 units), and the response was sustained for up to 8 weeks after treatment initiation.

TABLE 5
CDAI absolute and relative change at week 8 in ITT population

| | OC | | LOCF | |
|---|---|---|---|---|
| | Masitinib | Placebo | Masitinib | Placebo |
| N | 10 | 5 | 16 | 5 |
| Mean CDAI absolute change | −86.0 (128.9) | −13.2 (93.9) | −59.9 (112.2) | −13.2 (93.9) |
| from baseline (SD) | | | | |
| 95% CI (mean) | [−178.2; 6.2] | [−129.8; 103.4] | [−119.7; −0.1] | [−129.8; 103.4] |
| Median | −101.0 | 44.0 | −64.5 | 44.0 |
| P-value | 0.270 (W) | | 0.563 (W) | |
| Mean CDAI relative change | −23.3% (36.1) | −4.6% (31.3) | −17.0% (31.2) | −4.6% (31.3) |
| from baseline (SD) | | | | |
| 95% CI (mean) | [−49.1; 2.5] | [−43.5; 34.3] | [−33.6; −0.4] | [−43.5; 34.3] |
| Median | −26.9% | 12.6% | −21.4% | 12.6% |

| | | |
|---|---|---|
| P-value | 0.245 (W) | 0.342 (W) |

### CDAI Response According to Previous Treatment

An exploratory analysis was performed according to the previous treatment received:
L1: patients naive or having received previous corticosteroids
L2: patients having received previous azathioprine (Immurel) or immunotherapy alone
L3: patients having received previous biological agent (all anti-TNF therapy)

A total of 3 patients (1 treated with masitinib and 2 treated with placebo) were treatment naïve or had previously been treated with corticosteroids only. The results, displayed in Table 6, showed that one patient (100%) under masitinib had a clinical enhanced response (i.e. at least 100 points decrease), as compare to none in the placebo group. A difference between these cohorts was also observed in terms of the average CDAI relative change from baseline, i.e. −61% versus +18%, respectively. These results indicate that masitinib is active in a first-line treatment setting with patients that are treatment naïve to immunotherapy or biologic agents.

Two patients, previously treated with azathioprine (Immurel) or immunotherapy alone, were allocated to the masitinib group, one of whom presented with a clinical CDAI response at week 8. Overall there was an average decrease of CDAI score relative to baseline of 15%. These results indicate that masitinib is active in a second-line treatment setting with patients that have received at least one previous line of immunotherapy.

Most of the patients included in the study were previously treated with a biological agent (in this case anti-TNF therapy). At week 8, 5/7 (71%) masitinib treated patients reached a CDAI response versus 1/3 (33%) placebo treated patient. Of these, 4/7 (57%) masitinib patients reached an enhanced CDAI response versus 1/3 (33%) placebo treated patients. These results indicate that masitinib is active in a third-line treatment setting with patients that have received at least one prior line of biologics.

Thus, in one particular embodiment, because the response obtained with anti-TNF therapy is not maintained for a long time, masitinib benefits patients' refractory to biologics, especially after failure to anti-TNF therapy.

TABLE 6
CDAI response at week 8, according to previous treatment for Crohn's disease, ITT population, OC dataset

| | | | | | Patients previously treated | | | | | | |
|---|---|---|---|---|---|---|---|---|---|---|---|
| | Patients naive or previously | | | | with azathioprine (Immurel) | | | Patients previously treated with | | | |
| | treated with corticosteroids (L1) | | | | or immunotherapy (L2) | | | biological agent (L3) | | | |
| | Masitinib | | Placebo | | Masitinib | | Placebo | Masitinib | | Placebo | |
| N | 1 | | 2 | | 2 | | 0 | 7 | | 3 | |
| CDAI | 1 | (100.0%) | 0 | (0.0%) | 1 | (50.0%) | — | 5 | (71.4%) | 1 | (33.3%) |
| response* | | | | | | | | | | | |
| 95% CI | [20.7%; 100.0%] | | [0.0%; 65.8%] | | [9.5%; 90.5%] | | — | [35.9%; 91.8%] | | [6.1%; 79.2%] | |
| P-value | 0.333 | (F) | | | — | | | 0.500 | (F) | | |
| CDAI | 1 | (100.0%) | 0 | (0.0%) | 0 | (0.0%) | — | 4 | (57.1%) | 1 | (33.3%) |
| enhanced | | | | | | | | | | | |
| response** | | | | | | | | | | | |
| 95% CI | [20.7%; 100.0%] | | [0.0%; 65.8%] | | | | — | [25.0%; 84.2%] | | [6.1%; 79.2%] | |
| P-value | 0.333 | (F) | | | — | | | 1.000 | (F) | | |
| Mean CDAI | −153.0 | (.) | 51.5 | (10.6) | −50.0 | (50.9) | — | −86.7 | (152.7) | −56.3 | (103.0) |
| absolute | | | | | | | | | | | |
| change from | | | | | | | | | | | |
| baseline (SD) | | | | | | | | | | | |
| 95% CI | | | [−43.8; 146.8] | | [−507.4; 407.4] | | — | [−228.0; 54.5] | | [−312.2; 199.5] | |
| (mean) | | | | | | | | | | | |
| Median | −153.0 | | 51.5 | | −50.0 | | — | −103.0 | | −68.0 | |
| P-value | 0.540 | (W) | | | — | | | 0.820 | (W) | | |
| Mean CDAI | −61.0% | (.) | 17.6% | (6.0) | −14.9% | (13.0) | — | −20.3% | (40.7) | −19.3% | (33.6) |
| relative | | | | | | | | | | | |
| change from | | | | | | | | | | | |
| baseline (SD) | | | | | | | | | | | |

| 95% CI | | | [−36.1; 71.2] | [−132.0; 102.1] | — | [−57.9; 17.3] | | [−102.8; 64.2] |
|---|---|---|---|---|---|---|---|---|
| (mean) | | | | | | | | |
| Median | −61.0% | | 17.6% | −14.9% | — | −28.0% | | −16.0% |
| P-value | 0.540 | (W) | | — | | 0.820 | (W) | |

*Response: “at least 70 pts decrease”;
**enhanced response: “at least 100 pts decrease”

Overall, these results show that masitinib is active in patients with moderate to severe Crohn's disease that are either naïve or refractory to immunotherapy and/or biologics.

### CDAI Response According to Disease Activity at Baseline

Patients were analyzed according to their disease severity (i.e. with CDAI>350 at baseline) for disease response, absolute and relative changes at week 8.

The results displayed in Table 7 showed that patients with CDAI>350 at baseline were better treatment responders.

After 2 months of masitinib treatment, all the six evaluated patients (100%) with CDAI>350 at baseline responded, of whom four (67%) had an enhanced response.

Analysis of CDAI absolute and relative changes showed a significant improvement of mean CDAI in patients under masitinib.

TABLE 7
CDAI response, absolute and relative changes at week 8, patients with
CDAI >350 at baseline, ITT population

| | All | | CDAI >350 | |
|---|---|---|---|---|
| | Masitinib | Placebo | Masitinib | Placebo |
| N | 10 | 5 | 6 | 2 |
| CDAI response | 7 (70.0%) | 1 (20.0%) | 6 (100.0%) | 0 (0.0%) |
| 95% CI | [39.7%; 89.2%] | [3.6%; 62.4%] | [61.0%; 100.0%] | [0.0%; 65.8%] |
| P-value | 0.119 (F) | | 0.036 (F) | |
| CDAI enhanced response | 5 (50.0%) | 1 (20.0%) | 4 (66.7%) | 0 (0.0%) |
| 95% CI | [23.7%; 76.3%] | [3.6%; 62.4%] | [30.0%; 90.3%] | [0.0%; 65.8%] |
| P-value | 0.580 (F) | | 0.429 (F) | |
| CDAI absolute change from | −86.0 (128.9) | −13.2 (93.9) | −152.3 (78.5) | −8.0 (84.9) |
| baseline Mean (SD) | | | | |
| 95% CI (mean) | [−178.2; 6.2] | [−129.8; 103.4] | [−234.7; −70.0] | [−770.4; 754.4] |
| Median | −101.0 | 44.0 | −116.5 | −8.0 |
| P-value | 0.270 (W) | | 0.067 (W) | |
| CDAI relative change from | −23.3 (36.1) | −4.6 (31.3) | −38.8 (18.3) | −1.7 (20.2) |
| baseline Mean (SD) | | | | |
| 95% CI (mean) | [−49.1; 2.5] | [−43.5; 34.3] | [−58.0; −19.7] | [−183.2; 179.7] |
| Median | −26.9 | 12.6 | −29.5 | −1.7 |
| P-value | 0.245 (W) | | 0.067 (W) | |

*Response: “at least 70 pts decrease”;
**enhanced response: “at least 100 pts decrease”
(F) Fisher's exact test,
(W) Wilcoxon test

Patients with CDAI>350 at baseline responded well to masitinib at week 8, with 6/6 (100%) masitinib treated patients versus 0/2 (0%) placebo treated patients responding to treatment. Complete remission was observed in one patient (10%) presenting with CDAI>350 at baseline.

### Evolution of C—Reactive Protein Rate

CRP improvement was assessed in overall population and in patients previously treated with anti-TNF at week 8 (Table 8).

TABLE 8
CRP absolute and relative changes at week 8,
OC dataset, ITT population

| | All | |
|---|---|---|
| | Masitinib (N = 8) | Placebo (N = 5) |
| CRP mean absolute change ± | −19.7 ± 21.8 | 23.2 ± 38.9 |
| SD (mg/L) | | |
| 95% CI (mean) | [−38.0; −1.5] | [−25.1; 71.5] |
| Median | −19.0 | 0.0 |
| P value | 0.124 (W) | |
| CRP mean relative change ± SD | −27.4% ± 55.5 | 49.3% ± 107.7 |
| 95% CI (mean) | [−73.8; 19.0] | [−84.4; 183.0] |
| Median | −46.5% | 0.0% |
| P value | 0.164 (W) | |

Patients treated with masitinib had a decline from baseline in the serum levels of C-reactive protein, whereas patients in the placebo group did not.

Analysis of the average change relative to baseline in CRP level showed an important decrease in generalized inflammation for patients treated with masitinib (−27% versus +49% placebo at week 8).

A decline in CRP level was also observed in patients previously treated with anti-TNF, with a 30% decrease from baseline in masitinib patients as compared to an increase of 28% from baseline in patients given placebo (−30% versus +28%, respectively).

### Corticosteroids Decrease as Efficacy Criteria

Fifteen patients were on corticosteroids prior to study entry. Among the 8 patients who maintained corticosteroids during the study, the dose was decreased during study for 3/6 masitinib patients (50%) versus 0/2 placebo (0%). This indicates that masitinib may act as steroid-sparing therapy for reducing a patient's corticosteroid intake.

### Safety Results

All masitinib doses were well tolerated throughout the 8 weeks of observation.

The rate of patients who experienced adverse events during the treatment was similar in both groups: 94% of masitinib treated patients experienced at least one adverse event (AE) compared with 100% in the placebo treated patients. The most frequent adverse events under masitinib were vomiting (33% masitinib, 0% placebo), asthenia (33% masitinib, 40% placebo), nausea (28% masitinib, 0% placebo) and abdominal pain (22% masitinib, 0% placebo). During this period, 10 patients had a serious adverse event: 8 in the masitinib group (44%) and 2 in the placebo group (40%). There was no death.

Thus, oral masitinib is observed to be well-tolerated in patients with moderate-to-severe active Crohn's disease when administered at 4.5 or 6 mg/kg/day in two daily intakes.

### Study Conclusion

Efficacy results show that patients suffering from moderate-to-severe active Crohn's disease, including patients previously treated with anti-TNF therapy, demonstrated meaningful improvement in CDAI responses and associated inflammation.

Results from this study show that there is a satisfactory balance between the benefit of masitinib and its tolerance.

### Example 2

### Phase IIb/III—Clinical Study Protocol

### Study Design

The objective of the Phase IIb/III study is to compare the efficacy and/or safety of masitinib to placebo in the treatment of moderate Crohn's disease in patients intolerant or with unsatisfactory response to immunosuppressive drugs and/or TNF-inhibitors.

A pharmacogenomic study is also to be performed in order to define efficacy or safety genomic predictive criteria.
Study design: Double-blind, placebo-controlled, randomized, 2 parallel groups, 2:1 ratio, multicenter study
Diagnosis: Adult outpatients with moderate active Crohn's disease intolerant or with unsatisfactory response to immunosuppressive drugs and/or TNF-inhibitors
Study treatment: Masitinib, inhibitor of tyrosine kinase, 100 and 200 mg tablets
Associated product: Matching placebo, 100 and 200 mg tablets
Duration of treatment: 12 weeks (possible extension period for patient with clinical benefit)

Masitinib is supplied as 100 and 200 mg non-divisible capsule-shaped orange coated tablets. Inactive ingredients are microcrystalline cellulose, povidone, crospovidone, magnesium stearate and Opadry orange coating agent.

### Randomization

Patients will be randomized in 2 groups:
Group 1: 225 patients will receive masitinib at 3 mg/kg/day during 4 weeks, then 4.5 mg/kg/day during 4 weeks and then 6 mg/kg/day, and [azathioprine or 6-mercaptopurine or methotrexate]
Group 2: 225 patients will receive placebo with the same administration plan as masitinib and [azathioprine or 6-mercaptopurine or methotrexate]

Treatment allocation will use a 1:1 ratio design.

At randomization, patients will be centrally allocated to one of the 2 treatment groups by IWRS according to minimization method.

### Stratification

Minimization will be done on:
Previous treatment:
   No previous exposure with anti-TNF alpha treatment
   Previous exposure with anti-TNF alpha treatment
CDAI score at baseline

Randomization system is designed in order to have 50% of the patients never treated with anti-TNF alpha, and 50% of the patients with previous exposure with anti-TNF alpha treatment.

### Exposure Duration & Maximum Exposure

At week 12, in case of clinical response (CDAI decrease≧70) and if recommended by the investigator, patients will have the possibility to enter an extension period. Patients successfully treated with masitinib will be allowed to continue their treatment with extension visits performed every 12 weeks until clinical response remains favorable.

Hence, patients will be treated in a double blinded manner until the clinical database is locked. As soon as the treatment groups will be known, patients treated with placebo will be withdrawn from the study.

### Concomitant Treatments for Patients Receiving Azathioprine or 6-Mercaptopurine or Methotrexate

At randomization, azathioprine, 6-mercaptopurine, methotrexate must have been administered at a stable dose for a minimum of 4 weeks prior to treatment initiation. Patients have to continue their immunomodulative treatment at stable dose during the study.

Azathioprine (at least 2 mg/kg), 6-mercaptopurine (at least 1 mg/kg) or methotrexate (at least 15 mg/kg) will be administered according to usual practice. If azathioprine or 6-mercaptopurine or methotrexate were stopped prior to study treatment initiation, patients will receive masitinib or placebo alone in each treatment group respectively. Treatment allocation will use a 2:1 ratio design.

### Dose of Study Treatment

Dose of study treatment according to patient's weight are indicated in the tables below. For morning administration, tablets should be taken during breakfast. In case of nausea, the administration can take place during lunch. For the evening administration, tablets should be taken during dinner.

TABLE 9
Dose of study treatment (mg) according to
patient's weight (3 mg/kg/day)

| | | 3 mg/kg/day | | |
|---|---|---|---|---|
| | | Daily dose | | |
| Patient's weight in kg | | (mg) | Morning* (mg) | Evening** (mg) |
| >49.9 | ≦55.5 | 100 | | 100 |
| >55.5 | 77.7 | 200 | 100 | 100 |
| >77.7 | 99.9 | 300 | 100 | 200 |
| >99.9 | | 400 | 200 | 200 |

TABLE 10
Dose of study treatment (mg) according to
patient's weight (4.5 mg/kg/ day)

| | | 4.5 mg/kg/day | | |
|---|---|---|---|---|
| | | Daily dose | | |
| Patient's weight in kg | | (mg) | Morning* (mg) | Evening** (mg) |
| >49.9 | ≦55.5 | 200 | 100 | 100 |
| >55.5 | 77.7 | 300 | 100 | 200 |
| >77.7 | 99.9 | 400 | 200 | 200 |
| >99.9 | | 500 | 200 | 200 + 100 |

TABLE 11
Dose of study treatment (mg) according to
patient's weight (6 mg/kg/day)

| | | 6 mg/kg/day | | |
|---|---|---|---|---|
| | | Daily dose | | |
| Patient's weight in kg | | (mg) | Morning* (mg) | Evening** (mg) |
| >49.9 | ≦55.5 | 300 | 100 | 200 |
| >55.5 | 77.7 | 400 | 200 | 200 |
| >77.7 | 99.9 | 500 | 200 | 200 + 100 |
| >99.9 | | 600 | 200 + 100 | 200 + 100 |

### Steps for Dose Reduction

According to the current dose of study treatment or matching placebo, the steps for the dose reduction are as follows in Table 12:

| Current | 1^{st} dose | 2^{nd} dose | 3^{rd} dose | 3^{rd} dose |
|---|---|---|---|---|
| dose | reduction | reduction | reduction | reduction |
| 6 mg/kg/day | 4.5 mg/kg/day | 3 mg/kg/day | 1.5 mg/kg/ | STOP |
| | | | day | |
| 4.5 mg/kg/day | 3 mg/kg/day | 1.5 mg/kg/day | STOP | — |
| 3 mg/kg/day | 1.5 mg/kg/day | STOP | — | — |
| 1.5 mg/kg/day | STOP | — | — | — |

No dose escalation will be authorized for patients who have had a dose reduction for safety reasons.

### Main Inclusion Criteria

1. Patient with at least a 6-month history of Crohn's disease from diagnosis.
2. Patient with Crohn's disease based on clinical history and radiologic or endoscopic findings within the previous 24 months. Patients with active disease following surgical resection must have radiological or endoscopic confirmation of Crohn's disease post-surgery.
3. Patient with a CDAI (Crohn's disease activity index) score at baseline between 220 and 450 ([220; 450]).
4. Patient intolerant or with unsatisfactory response to azathioprine or 6-mercaptopurine or methotrexate and/or TNF-inhibitors.
Patient with unsatisfactory response is defined as patient with active Crohn's disease (CDAI>=220) after at least 3 months under stable dose of treatment. Patient intolerant is defined as a patient who has had to discontinue treatment at any time for tolerability or safety reason.
5. Patient with active Crohn's disease confirmed by at least one of the following criteria:
   level of C-reactive protein>5 mg/L within 2 weeks prior to study treatment initiation;
   fecal calprotectin level>250 μg within 2 weeks prior to study treatment initiation;
   ulcers demonstrated at colonoscopy within 12 weeks prior to study treatment initiation;
   small intestine or colonic inflammation defined as post-contrast parietal relative contrast enhancement after gadolinium injection and/or the presence of deep ulcerations on MRI or CT within 6 weeks prior to study treatment initiation;
6. Male or female patient aged 18 to 75 years, weight>50 kg, BMI between 18 and 35 kg/m².

### Main Exclusion Criteria

1. Patient with less than 3 months exposure to azathioprine or 6-mercaptopurine or methotrexate;
2. Patient treated with biological agents other than anti-TNF alpha;
3. Patient with known active or draining fistula;
4. Patient currently receiving nasogastric/nasoenteric tube feeding, an elemental/polymeric diet or total parenteral nutrition e.g. liquid diet and oral supplements, must remain stable during the treatment phase (baseline to week 4);
5. Patient likely to require emergency surgery for persistent intestinal obstruction, bowel perforation, uncontrolled bleeding or abdominal abscess infection, in the investigator's opinion and patients who have undergone bowel surgery within the 3 months prior to study treatment initiation;
6. Patient with previous proctocolectomy or with symptomatic stricture or with abscess;
7. Patient with a clinically relevant short bowel syndrome, in the opinion of the investigator;
8. Patient whose symptoms are believed to be largely due to the presence of fixed fibrotic strictures in the opinion of the investigator;
9. Patient with a history of malignant neoplastic diseases of the bowel including diseases occurring more than 5 years before the inclusion in the study.

### Endpoints

### Primary Endpoint

Enhanced clinical response, defined as a decrease in CDAI (Crohn's Disease Activity Index)≧100 at week 12 (W meaning week).
Main secondary endpoints include:
Clinical remission, defined as CDAI<150 and absence of surgery at W4, W8 and W12;
Clinical response defined as a decrease in CDAI≧70 at W4, W8 and W12;
Enhanced clinical response, defined as a decrease in CDAI≧100 at W4 and W8;
Cumulative clinical response;
Cumulative enhanced clinical response;
Cumulative clinical remission;
C-reactive protein change at W4, W8 and W12;
Corticosteroids consumption at W4, W8, W9, W10, W11 and W12;
Quality of Life;
   Absolute and relative change from baseline in IBDQ (Inflammatory Bowel Disease Questionnaire) scores at W4, W8 and W12;
   Absolute and relative change from baseline in FACIT-Fatigue (Functional Assessment of Chronic Illness Therapy-Fatigue score at W4, W8 and W12;
   Absolute and relative change from baseline in W12 IBD disability index (Inflammatory Bowel Disease disability index) at W4, W8 and W12.

1. A method for treating Crohn's disease in a subject in need thereof, comprising administering to said subject a therapeutically effective amount of masitinib or a pharmaceutically acceptable salt or solvate thereof, wherein said subject has moderately-severe active Crohn's disease with a Crohn's Disease Activity Index (CDAI) superior to 350 (CDAI>350) at baseline.

2. The method according to claim 1, wherein the pharmaceutically acceptable salt or solvate of masitinib is masitinib mesilate.

3. The method according to claim 1, wherein the therapeutically effective amount of masitinib or a pharmaceutically acceptable salt or solvate thereof ranges from about 1.0 mg/kg/day to about 10 mg/kg/day (mg per kg bodyweight per day).

4. The method according to claim 1, wherein the therapeutically effective amount of masitinib or a pharmaceutically acceptable salt or solvate thereof is administered in two daily intakes.

5. The method according to claim 1, wherein said therapeutically effective amount of masitinib or a pharmaceutically acceptable salt or solvate thereof is administered at a dose of 1.5, 3.0, 4.5, 6.0, 7.5, or 9.0 mg/kg/day.

6. The method according to claim 1, wherein the therapeutically effective amount of masitinib or a pharmaceutically acceptable salt or solvate thereof is initially administered at a dose of 3 mg/kg/day during 4 weeks, then 4.5 mg/kg/day during 4 weeks, and at 6 mg/kg/day thereafter, with each switch being subjected to toxicity controls.

7. The method of claim 1, wherein said subject is selected among one of the following groups of patients:
a group of patients who never received any therapy for Crohn's disease or have previously received controller medications selected from aminosalicylate or corticosteroid agents.
a group of patients who have previously received azathioprine, 6-mercaptopurine, methotrexate, or at least one prior course of an immunosuppressive drug.
a group of patients refractory to azathioprine, 6-mercaptopurine, methotrexate, or at least one previous course of immunosuppressive treatment for Crohn's disease.
a group of patients who have previously received adalimumab, certolizumab pegol, infliximab, golimumab, natalizumab, vedolizumab, or one at least one prior course of biologic agent and/or tumor necrosis factor inhibitor.
a group of patients refractory to adalimumab, certolizumab pegol, infliximab, golimumab, natalizumab, vedolizumab, or at least one previous course of biologic agent and/or tumor necrosis factor inhibitor treatment for Crohn's disease.

8. The method of claim 1, wherein the subject is naïve to treatments for Crohn's disease, or wherein Crohn's disease relapsed after at least one treatment for Crohn's disease, or after two or more treatments for Crohn's disease.

9. The method according to claim 1, wherein the subject is refractory to treatments for Crohn's disease.

10. The method according to claim 1, wherein the subject has a concomitant treatment with corticosteroids and wherein masitinib acts as steroid-sparing therapy for reducing the intake of corticosteroids of said subject.

11. The method according to claim 1, comprising administering to said subject a therapeutically effective amount of masitinib or a pharmaceutically acceptable salt or solvate thereof in combination with a therapeutically effective amount of at least one pharmaceutically active ingredient for treatment of Crohn's disease.

12. The method according to claim 1, comprising administering to said subject a therapeutically effective amount of masitinib or a pharmaceutically acceptable salt or solvate thereof in combination with a therapeutically effective amount of at least one pharmaceutically active ingredient for treatment of Crohn's disease, wherein said pharmaceutically active ingredient is selected from: aminosalicylates, corticosteroids, immunotherapy agents, biologic agent or tumor necrosis factor alpha inhibitors.

13. The method according to claim 1, comprising administering to said subject a therapeutically effective amount of masitinib or a pharmaceutically acceptable salt or solvate thereof in combination with a therapeutically effective amount of at least one aminosalicylate or corticosteroid, wherein said at least one aminosalicylate or corticosteroid is selected from: balsalazide, olsalazine, mesalamin, sulfasalazine, prednisolone, and dexamethasone.

14. The method according to claim 1, comprising administering to said subject a therapeutically effective amount of masitinib or a pharmaceutically acceptable salt or solvate thereof in combination with a therapeutically effective amount of at least one immunotherapy agent, wherein said at least one immunotherapy agent is selected from: azathioprine, 6-mercaptopurine, and methotrexate.

15. The method according to claim 1, comprising administering to said subject a therapeutically effective amount of masitinib or a pharmaceutically acceptable salt or solvate thereof in combination with a therapeutically effective amount of at least one biologic agent or tumor necrosis factor alpha inhibitor, wherein said at least one biologic agent is selected from the group consisting of: adalimumab, certolizumab pegol, infliximab, golimumab, natalizumab, and vedolizumab.

16. A pharmaceutical composition comprising masitinib or a pharmaceutically acceptable salt or solvate thereof, and a pharmaceutically active ingredient for treatment of Crohn's disease, in combination with at least one pharmaceutically acceptable carrier, wherein said pharmaceutically active ingredient is selected from: azathioprine, 6-mercaptopurine, methotrexate, adalimumab, certolizumab pegol, infliximab, golimumab, natalizumab, vedolizumab, balsalazide, olsalazine, mesalamin, sulfasalazine, prednisolone, dexamethasone, and mixtures thereof.

17. A medicament comprising masitinib or a pharmaceutically acceptable salt or solvate thereof and at least one pharmaceutically active ingredient for treatment of Crohn's disease, wherein said pharmaceutically active ingredient is selected from: azathioprine, 6-mercaptopurine, methotrexate, adalimumab, certolizumab pegol, infliximab, golimumab, natalizumab, vedolizumab, balsalazide, olsalazine, mesalamin, sulfasalazine, prednisolone, dexamethasone, and mixtures thereof.

18. A kit of part comprising, in a first part, masitinib or a pharmaceutically acceptable salt or solvate thereof, and in a second part, at least one pharmaceutically active ingredient for treatment of Crohn's disease selected from: azathioprine, 6-mercaptopurine, methotrexate, adalimumab, certolizumab pegol, infliximab, golimumab, natalizumab, vedolizumab, balsalazide, olsalazine, mesalamin, sulfasalazine, prednisolone, dexamethasone, and mixtures thereof.
